(19) United States
(12) Patent Application Publication
(10) Pub. No.: US 2025/0099332 A1
(43) Pub. Date: Mar. 27, 2025

(54) A CONNECTOR

(71) Applicant: Oribiotech LTD, London, Great London (GB)

(72) Inventors: Farlan Veraitch, London, Greater London (GB); William Raimes, London, Greater London (GB); Stephen Shapka, London, Greater London (GB); Arman Amini, London, Greater London (GB); Richard Smith, Hertfordshire (GB); Neil Litten, Hertfordshire (GB); Julian Brandon-Jones, Hertfordshire (GB)

(21) Appl. No.: 18/832,409

(22) PCT Filed: Jan. 20, 2023
(86) PCT No.: PCT/GB2023/050123

§ 371 Date: Jul. 23, 2024

(30) Foreign Application Priority Data
Jan. 24, 2022 (GB) 2200869.2

(51) Int. Cl. A61J 1/20

(52) U.S. Cl. CPC A61J 1/2048

(57) ABSTRACT

The present disclosure concerns a connector including a housing, a hollow needle, an actuating mechanism, and a locking mechanism. The housing including a first end and a second end. The hollow needle mounted, at least partially, within the housing. A first end of the hollow needle being connected or connectable to a first receptacle and a second end of the hollow needle facing a pierceable seal. The actuating mechanism configured to cause relative movement between the hollow needle and the pierceable seal to enable the hollow needle to pierce the pierceable seal. The locking mechanism, in a first configuration, prevents the actuating mechanism from causing relative movement between the hollow needle and the pierceable seal, and in a second configuration, permits the actuating mechanism to cause relative movement between the hollow needle and the pierceable seal.

### CROSS REFERENCE TO RELATED APPLICATIONS

This application is a national phase entry under 35 U.S.C. § 371 of International Patent Application PCT/GB2023/050123, filed Jan. 20, 2023, designating the United States of America and published as International Patent Publication WO 2023/139383 A1 on Jul. 27, 2023, which claims the benefit under Article 8 of the Patent Cooperation Treaty to Great Britain Patent Application Serial No. 2200869.2, filed Jan. 24, 2022.

### TECHNICAL FIELD

The disclosure relates to a connector, for example, an aseptic connector. More particularly, the disclosure relates to a connector for introducing material to, or extracting material from, at least one receptacle. The connector described herein may form part of a biological processing apparatus, such as a cell and/or gene therapy manufacture apparatus.

### BACKGROUND

Biological processing methodologies, particularly those used in the manufacture of cell and/or gene therapy products, are often complex and require manual or semi-automated steps across multiple devices. The systems used in various steps, also referred to as unit operations, of manufacturing cell-based therapeutic products (CTP) may include a plethora of devices for various functions. These various functions can include, for example, cell collection, isolation, selection, expansion, proliferation, washing, storage or harvesting. The unit operations can vary significantly based on the manufacturing model (i.e., autologous or allogenic), the type of cell, the intended purpose, amongst other factors. Additionally, cells are “living” entities and are sensitive to even the most minor manipulations or changes in conditions, particularly during transfer between various devices.

The culture or processing of cells typically requires the use of a device to hold the cells in an appropriate culture medium for proliferation. Such known devices include shaker flasks, roller bottles, T-flasks, bags, bioreactors and the like. Subsequently, an addition or extraction step may be desirable to, or from, the device. For example, it may be desirable to provide an addition of culture medium, culture nutrients, cytokines, genetic modification agents, or like materials, in order to support the culture or processing methodology. Equally, it may be desirable to provide an extraction of exhausted culture medium—such as in a “washing” step—or to take a sample from the device.

As such, there is a need for connecting one device to another device during biological processing methodologies, such as cell and/or gene therapy manufacture, in order to enable a fluid communication between devices. Moreover, it is often desirable to provide a sterile, or an aseptic, connection between such devices, as well as a sterile disconnection once the material transfer has been completed.

One solution is to provide a needle-based connector to enable communication through such a needle-based connector between two devices to be connected. However, one drawback of such needle-based connectors is the requirement to mitigate, or at least reduce, needle-stick injuries, as such injuries are not only harmful to a user, whom may be exposed to harmful biological agents, but also compromises sterility of the connection.

Therefore, it is an object of the present disclosure to address one of the disadvantages associated with known connectors, particularly aseptic needle-based connectors. In particular, although not exclusively, one object of the present disclosure is to mitigate, obviate or otherwise reduce needle-stick injuries when handling needle-based connection systems.

### BRIEF SUMMARY

According to one aspect of the present disclosure, there is provided a connector, for introducing or extracting material to or from at least one receptacle, comprising:
a housing, extending between a distal end and a proximal end;
a hollow needle mounted, at least partially, within the housing between the distal end and the proximal end, a first end of the hollow needle being connected or connectable to a first receptacle, and a second end of the hollow needle facing a pierceable seal;
an actuating mechanism configured to cause relative movement between the hollow needle and the pierceable seal to enable the hollow needle to pierce the pierceable seal, thereby forming a communication therethrough; and
a locking mechanism operable between a first configuration, in which the locking mechanism prevents the actuating mechanism from causing relative movement between the hollow needle and the pierceable seal, and a second configuration, in which the locking mechanism permits the actuating mechanism to cause relative movement between the hollow needle and the pierceable seal.

This provides the advantage that the hollow needle only pierces the pierceable seal upon operation of the locking mechanism. That is, in the first position, the hollow needle is prevented from piercing the pierceable seal. In the second position, once the locking mechanism is operated by the user or a machine, the hollow needle is permitted to pierce the pierceable seal and form a communication therethrough. Thus, needle-stick injuries may be mitigated, or at least reduced, during use of the needle-based connector.

In certain embodiments, the pierceable seal is a self-sealing seal. In certain embodiments, the pierceable seal is a septum seal. In certain embodiments, the pierceable seal is a hermetic seal. In certain embodiments, the housing comprises the pierceable seal. In other embodiments, a portion of the actuating mechanism may comprise the pierceable seal.

In certain embodiments, the actuating mechanism comprises a collapsible sleeve assembly, wherein at least part of the collapsible sleeve assembly is collapsible between the proximal end and the distal end of the housing to enable the hollow needle to pierce the pierceable seal, thereby forming a communication therethrough.

Thus, in certain embodiments, the locking mechanism is operable between a first configuration, in which the locking mechanism prevents collapsing of the collapsible housing, and a second position, in which the locking mechanism permits collapsing of the collapsible housing to enable the hollow needle to pierce the pierceable seal.

In certain embodiments, the collapsible sleeve assembly may include a portion of the housing. In certain embodiments, the housing, or a portion thereof, may be configured to cause collapsing of the collapsible sleeve assembly.

In certain embodiments, the collapsible sleeve assembly comprises a moveable sleeve portion and a stationary sleeve portion, the moveable sleeve portion being axially moveable, along a central longitudinal axis, with respect to the stationary sleeve portion so as to collapse with respect thereto.

Thus, in certain embodiments, the locking mechanism is operable between a first configuration, in which the locking mechanism prevents axial movement of the moveable sleeve portion, and a second position, in which the locking mechanism permits axial movement of the moveable sleeve portion to enable the hollow needle to pierce the pierceable seal.

In certain embodiments, the moveable sleeve portion comprises at least one actuatable element configured to be operably engaged by an activation system. In certain embodiments, the at least one actuatable element comprises a lug, a shoulder, a protrusion, a clasp, a coupling element, a fastener, a loop or the like. The at least one actuatable element may be hollow. In some embodiments, the moveable sleeve portion comprises a pair of actuatable elements.

In certain embodiments, the moveable sleeve portion comprises the pierceable seal. In some embodiments, the moveable sleeve portion comprises the pierceable seal at a distal end thereof, which may be adjacent to, or corresponding with, the distal end of the housing.

In certain embodiments, the locking mechanism comprises a clip slidably disposed within a lateral slot of the moveable sleeve portion, wherein, in the first position, the moveable clip is engaged with a portion of the stationary sleeve portion, and in the second position, the moveable clip is disengaged from the portion of the stationary sleeve portion.

In certain embodiments, the locking element comprises the clip slidably disposed within a hollow actuatable element of the moveable sleeve portion.

In certain embodiments, the clip is biased toward, or into, the first position by a biasing element.

This provides the advantage that, following an actuation of the actuating mechanism and thus the hollow needle, the locking mechanism reverts to the first, or locked, position, thereby mitigating, or at least reducing, needle-stick injuries.

In certain embodiments, the clip comprises at least one spring arm. In certain embodiments, the clip is provided with a pair of spring arms. The or each spring arm may be deflectable or resilient.

In certain embodiments, the hollow needle is a double-ended hollow needle. The double-ended hollow needle may include a piercing end at each end thereof.

In certain embodiments, the stationary sleeve portion comprises a pierceable seal, and wherein the double-ended hollow needle is disposed between each pierceable seal. That is, in certain embodiments, the double-ended hollow needle is disposed between the pierceable seal of the moveable sleeve portion and the pierceable of the stationary sleeve portion. That is, in certain embodiments, each end of the double-ended hollow needle faces a pierceable seal.

In certain embodiments, the actuating mechanism is configured to cause relative movement between the double-ended hollow needle and each of the pierceable seals, to enable the double-ended hollow needle to pierce each of the pierceable seals, thereby forming a communication through each pierceable seal.

In certain embodiments, the actuating mechanism further comprises a collar operably coupled to the hollow needle, wherein the collar is axially moveable along a central longitudinal axis.

This provides the advantage that the translation of the hollow needle may be controlled along the central longitudinal axis.

In certain embodiments, the locking mechanism is operable between a first configuration, in which the locking mechanism prevents translation of the collar along the central longitudinal axis, and a second position, in which the locking mechanism permits translation of the collar along the central longitudinal axis.

In certain embodiments, the collar comprises at least one actuatable element configured to be operably engaged by an activation system. In certain embodiments, the at least one actuatable element comprises a lug, a shoulder, a protrusion, a clasp, a coupling element, a fastener, a loop or the like. The at least one actuatable element may be hollow. In some embodiments, the collar comprises a pair of actuatable elements.

In certain embodiments, the locking mechanism comprises a clip slidably disposed within a lateral slot of the collar, wherein, in the first position, the clip is engaged with a portion of the stationary sleeve portion, and in the second position, the moveable clip is disengaged from the portion of the stationary sleeve portion.

In certain embodiments, the collar comprises the clip slidably disposed within a hollow actuatable element of the collar.

In certain embodiments, the clip is biased toward the first position by a biasing element.

This provides the advantage that, following an actuation of the actuating mechanism and thus the hollow needle, the locking mechanism reverts to the first, or locked, position, thereby mitigating, or at least reducing, needle-stick injuries.

In certain embodiments, the clip comprises at least one spring arm. In certain embodiments, the clip is provided with a pair of spring arms. The or each spring arm may be deflectable or resilient.

According to another aspect of the present disclosure, there is provided a system, comprising:
a connector as described herein; and
an activation system configured to operably engage with the locking mechanism to enable operation of the locking mechanism between the first configuration and the second configuration.

In certain embodiments, the activation system is further configured to actuate the actuating mechanism to enable the hollow needle to pierce the pierceable seal and form a communication therethrough.

In certain embodiments, the activation system comprises at least one prong that is configured to operably engage the locking mechanism to enable operation of the locking mechanism between the first configuration and the second configuration.

In certain embodiments, the at least one prong of the activation system is configured to operably engage a clip disposed within a lateral slot of the moveable sleeve portion or the collar.

In certain embodiments, the at least one prong of the activation system is further configured to actuate the actuatable element of the moveable sleeve portion or the collar. In certain embodiments, the at least one prong of the activation system is further configured to translate the moveable sleeve portion or the collar by engaging the actuatable element.

In certain embodiments, the activation system includes a first prong configured to operably engage a clip disposed within a lateral slot of the moveable sleeve portion, and a second prong configured to operably engage a clip disposed within a lateral slot of the collar. The first prong may be provided as a pair of prongs to operably engage a pair of clips. The second prong may be provided as a second pair of prongs to operable engage a pair of clips.

In certain embodiments, the first prong is further configured to actuate the actuatable element of the moveable sleeve portion. In certain embodiments, the first prong of the activation system is further configured to translate the moveable sleeve portion by engaging the actuatable element. In certain embodiments, the second prong is further configured to actuate the actuatable element of the collar. In certain embodiments, the second prong of the activation system is further configured to translate the collar by engaging the actuatable element.

According to another aspect of the present disclosure, there is provided a method of connecting two receptacles, comprising:
providing a connector as described herein;
providing an activating system as described herein;
connecting the distal end of the housing of the connector to a first receptacle, such that the first receptacle is connected to the first end of the hollow needle;
detachably connecting the proximal end of the housing of the connector to a second receptacle comprising a pierceable seal, such that the pierceable seal of the connector is coaxially aligned with the pierceable seal of the second receptacle;
using the activating system to operably engage the locking mechanism of the connector so as to operate the locking mechanism from the first configuration to the second configuration;
actuating the hollow needle to pierce the pierceable seal of the connector and to pierce the pierceable seal of the second receptacle, thereby connecting the first receptacle and the second receptacle.

In certain embodiments, the method further comprises the steps of:
ceasing actuation of the hollow needle to cease piercing of the pierceable seal of the connector, thereby disconnecting the first receptacle and the second receptacle;
disengaging the activating system from the locking mechanism of the connector so as to operate the locking mechanism from the second configuration to the first configuration.

Reference will now be made to the drawings, which depict one or more embodiments described in this disclosure. However, it will be understood that other embodiments not depicted in the drawings fall within the scope of this disclosure. Like numbers used in the figures refer to like components, steps and the like. However, it will be understood that the use of a number to refer to a component in a given figure is not intended to limit the component in another figure labelled with the same, or indeed a different, number. The figures are presented for the purpose of illustration and are not limiting. Schematic drawings presented in the figures are not necessarily to scale.

### BRIEF DESCRIPTION OF THE DRAWINGS

FIG. 1 illustrates a perspective view of a connector according to an embodiment of the disclosure;

FIG. 2 illustrates an exploded view of the connector of FIG. 1;

FIG. 3 illustrates a detailed view of the actuating mechanism of the connector of FIG. 1;

FIG. 4 illustrates a cross-sectional view of the connector of FIG. 1;

FIGS. 5A-5C illustrate a perspective view of the connector of FIG. 1: FIG. 5A with the locking mechanism in the first configuration, FIG. 5B with the locking mechanism in the second configuration, and FIG. 5C following piercing of the first pierceable seal;

FIGS. 6A-6D illustrate a part-cutaway side view of the connector of FIG. 1: FIG. 6A with the locking mechanism in the first configuration, FIG. 6B with the locking mechanism in the second configuration, FIG. 6C following piercing of the first pierceable seal, and FIG. 6D following piercing of the second pierceable seal;

FIGS. 7A-7D illustrate a part-cutaway rear view of the connector of FIG. 1: FIG. 7A with the locking mechanism in the first configuration, FIG. 7B with the locking mechanism in the second configuration, FIG. 7C following piercing of the first pierceable seal, and FIG. 7D following piercing of the second pierceable seal;

FIGS. 8A and 8B illustrate a side-view of a clip of the locking mechanism illustrated in FIG. 1: FIG. 8A in the first configuration and FIG. 8B in the second configuration;

FIG. 9 illustrates an activating system for enabling operation of the connector of FIG. 1 during use; and

FIGS. 10A-10E illustrate the use of the activating system of FIG. 9 for enabling the operation of the connector of FIG. 1.

### DETAILED DESCRIPTION

The description example embodiments relate to a connector for introducing or extracting material to or from at least one receptacle. In particular, some embodiments relate to a connector that is an aseptic connector, or more particularly, a connector that maintains an aseptic environment during a material transfer. The terms “sterile” and “aseptic” are interchangeable. References to fluids in the detailed description are not intended to limit the scope of protection to such materials. As will be recognized by a person skilled in the art, fluids as described herein are merely an example of a suitable material for use with the described connector. Equally, reference may be made to a container, vacutainer or the like, however, such references in the detailed description are not intended to limit the scope of protection to such receptacles or vessels. As will be recognized by a person skilled in the art, containers, vacutainers or the like are described herein as mere examples of suitable receptacles.

Certain terminology is used in the following description for convenience only and is not limiting. The words ‘upper’ and ‘lower’ designate directions in the drawings to which reference is made and are with respect to the described component when assembled and mounted. The words ‘inner,’ ‘inwardly’ and ‘outer,’ and ‘outwardly’ refer to directions toward and away from, respectively, a designated centerline or a geometric center of an element being described (e.g., a central axis), the particular meaning being readily apparent from the context of the description. Further, the terms ‘proximal’ (i.e., nearer to) and ‘distal’ (i.e., away from) designate positions relative to an axis or a point of attachment.

Further, as used herein, the terms ‘connected,’ ‘affixed,’ ‘coupled’ and the like are intended to include direct connections between two members without any other members interposed therebetween, as well as, indirect connections between members in which one or more other members are interposed therebetween. The terminology includes the words specifically mentioned above, derivatives thereof, and words of similar import.

Further, unless otherwise specified, the use of ordinal adjectives, such as, ‘first,’ ‘second,’ ‘third’ etc., merely indicate that different instances of like objects are being referred to and are not intended to imply that the objects so described must be in a given sequence, either temporally, spatially, in ranking or in any other manner. Like reference numerals are used to depict like features throughout.

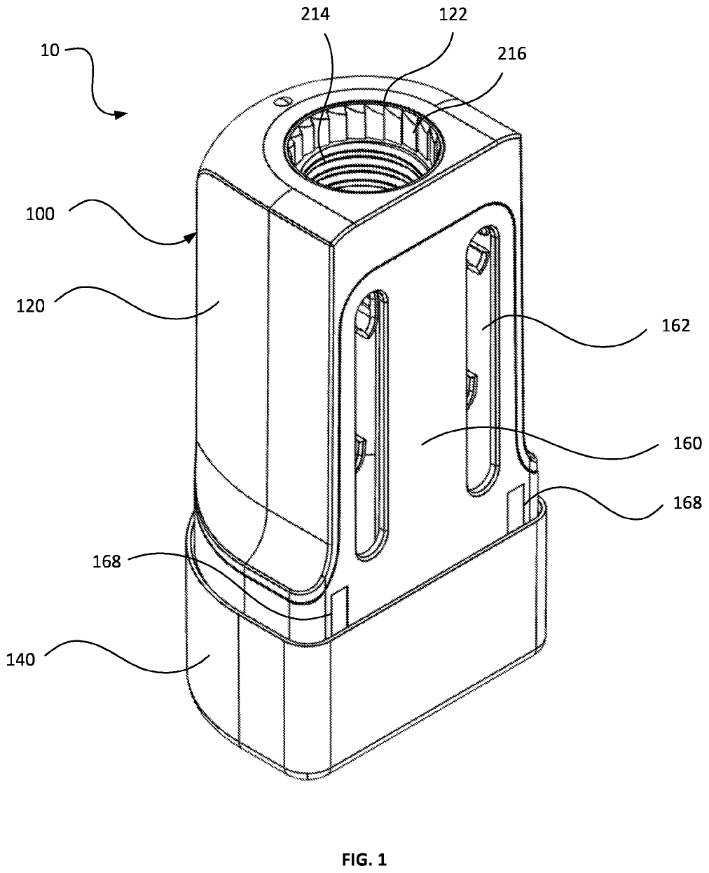

FIG. 1 illustrates a connector 10 including a housing 100 extending between a distal end and a proximal end. The housing 100 includes an outer cover 120 having flat side walls and a curved front wall, and a portion of the actuating mechanism, a stationary sleeve portion 160, as will be described hereinafter. The stationary sleeve portion 160 is provided with a pair of magnets 168 for cooperation with cooperating magnets on an activating system, as discussed further below, in order to provide a tactile feedback to a user during installation. The connector 10 also includes a cap 140 disposed at the proximal end of the outer cover 120 for enclosing a base of the connector 10. The connector 10 also includes, as part of a moveable sleeve portion as described hereinafter, a container-receiving portion 122 that is substantially cylindrical and includes an internally screw-threaded portion 214. The container-receiving portion 122 is also provided with anti-rotational threads 216 that are designed as a ratchet mechanism to interact with cooperating elements on a received container (not shown).

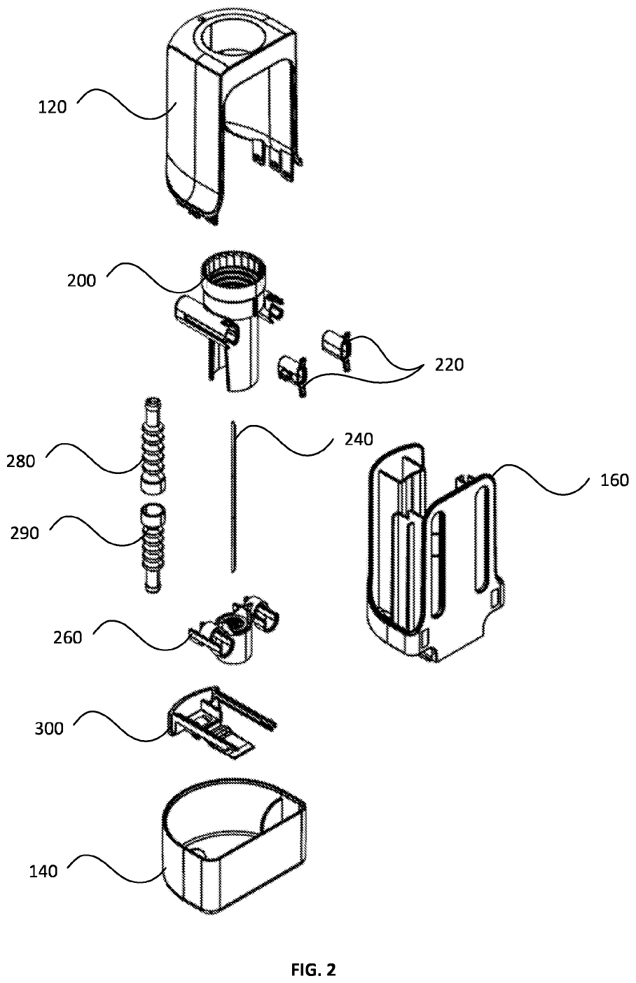

FIG. 2 illustrates an exploded view of the connector 10 of FIG. 1. As illustrated, the connector 10 is composed of an outer cover 120 and a cap 140 as described above. The connector 10 also includes a double-ended hollow needle 240 that is operably coupled to a collar 260 as described below. The hollow needle 240 is operated by virtue of an actuating mechanism, in this embodiment composed as a collapsible sleeve mechanism, including a moveable sleeve portion 200 and a stationary sleeve portion 160. The connector 10 is also provided with a sliding drawer 300, which is slidably coupled to the stationary sleeve portion 160 and covered by the cap 140 during transit of the connector 10.

Referring to FIGS. 2 to 4, the actuating mechanism is described as follows. There is provided the moveable sleeve portion 200 that includes a pair of laterally extending hollow lugs 202, 204, each including a laterally extending slot 206, 208. Within the hollow lugs 202, 204, there is provided a clip 220 that is slidably disposed within each hollow lug 202, 204. The clips 220 are each provided with a protrusion 222 that extends through each slot 206, 208 of the lugs 202, 204. As will become clear from the remaining description, the clips 220 act as a locking mechanism to prevent piercing of the pierceable seals by the needle 240 prior to use. The clips 220 are provided with a pair of spring arms 224, described in further detail below, which extend in the first configuration through recesses of the hollow lugs 202, 204 proximal to an open end 210, 212 thereof. The lugs 202, 204 are accessible, namely at the open end 210, 212 thereof, through a pair of longitudinally extending slots 162 of the stationary sleeve portion 160 (see FIG. 1).

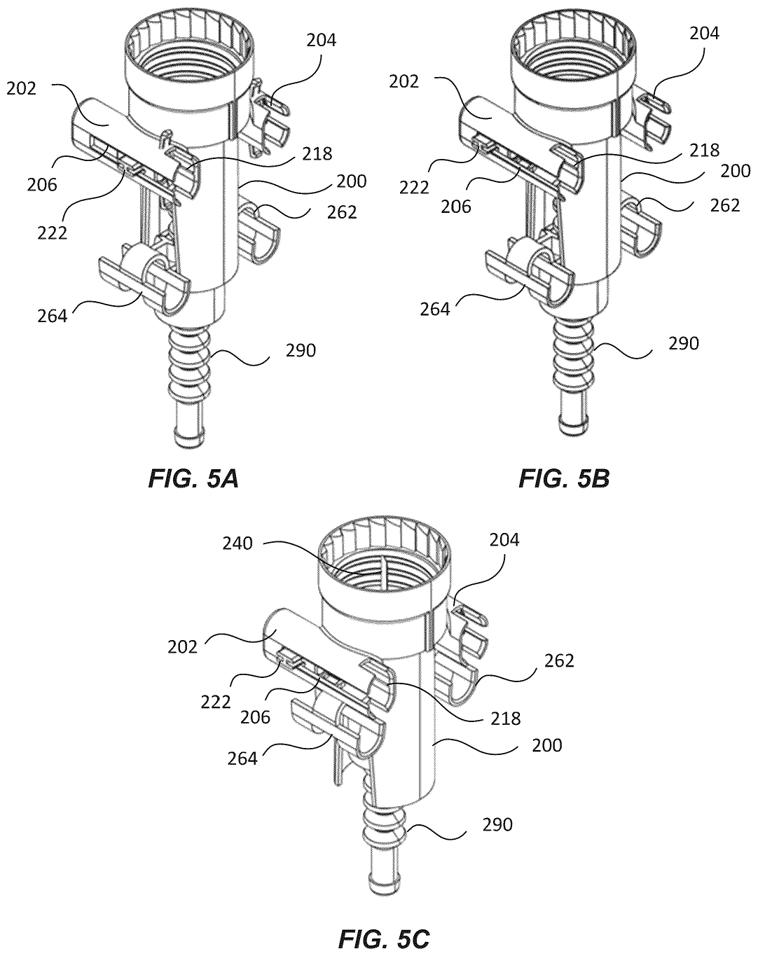

Furthermore, with further reference to FIGS. 5A-5C, the hollow lugs 202, 204 include a rib 218 having a variable thickness. In particular, the rib 218 has an increased thickness proximate the open end 210, 212, and a decreased thickness at the opposing end. In this way, tactile feedback is provided to the user when loading the connector onto an activating system as noted below.

There is also provided a collar 260 including a pair of laterally extending hollow lugs 262, 264. The collar 260 is operably coupled, for example, through a friction-fit, push-fit, adhesion or the like, to the hollow needle 240. The lugs 262, 264 are accessible through a pair of longitudinally extending slots 162 of the stationary sleeve portion 160 (see FIG. 1).

Although not shown, the lugs 262, 264 of the collar 260 may include clips in addition to, or as an alternative to, the clips 220 provided within the lugs 202, 204 of the moveable sleeve portion 200.

Additionally, as shown in FIGS. 1 and 2, there are provided two gaiters 280, 290, each gaiter 280, 290 enclosing a respective side of the hollow needle 240 and each side of the collar 260. That is, the first gaiter 280 extends from a first side of the collar 260 toward the distal end of the connector 10, and the second gaiter 290 extends from a second, opposing, side of the collar 260 toward the proximal end of the connector 10. The gaiters 280, 290 serve as sterility barriers during use, to ensure an aseptic environment for the needle 240.

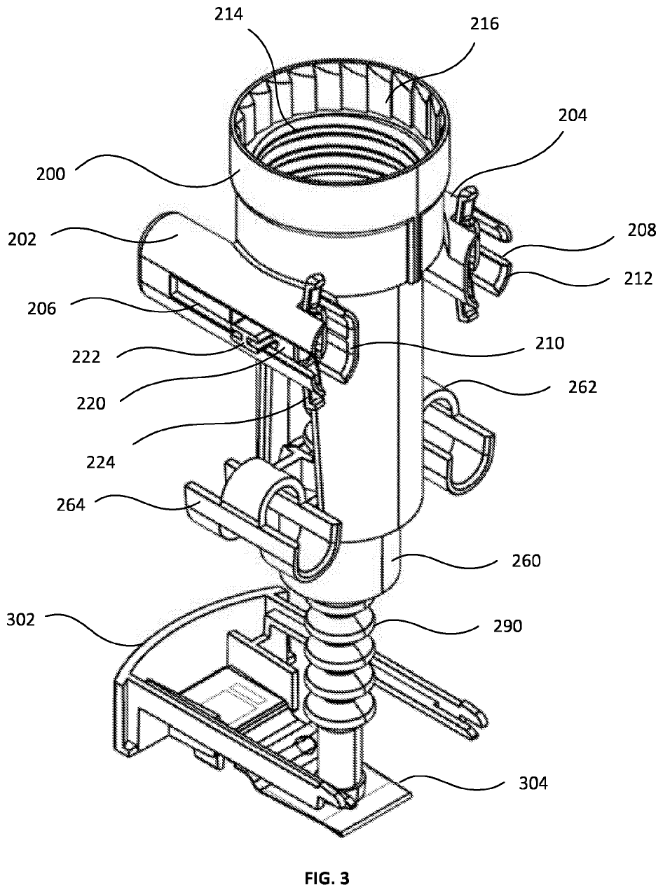

Finally, referring to FIGS. 2 and 3, there is provided a sliding drawer 300, which is slidably coupled to the stationary sleeve portion (see FIG. 2) and slidable with respect thereto. The sliding drawer 300 includes a front portion 302, from which a rail extends, which is received in a corresponding slot of the stationary sleeve portion during use. The sliding drawer 300 is coupled to an aseptic seal 304 by virtue of a heat weld. The aseptic seal 304 is provided as a folded piece of polymeric material that ensures sterility of a pierceable seal over which it is provided.

In addition to those features already described, the connector 10 notably includes a first pierceable seal 282 formed within the moveable sleeve portion 200 toward a distal end of the connector 10, and a second pierceable seal 292 formed within the stationary sleeve portion 160 (see FIG. 2) toward a proximal end of the connector 10. Each pierceable seal 282, 292 encloses the hollow needle 240 therebetween, such that each end of the hollow needle 240 faces a pierceable seal 282, 292 at respective ends. Prior to use, the hollow needle 240 faces each pierceable seal 282, 292 and does not protrude therethrough. Following actuation of the connector, the needle 240 is caused to pierce each pierceable seal 282, 292 to form a connection between two containers—each container disposed proximate to each pierceable seal 282, 292.

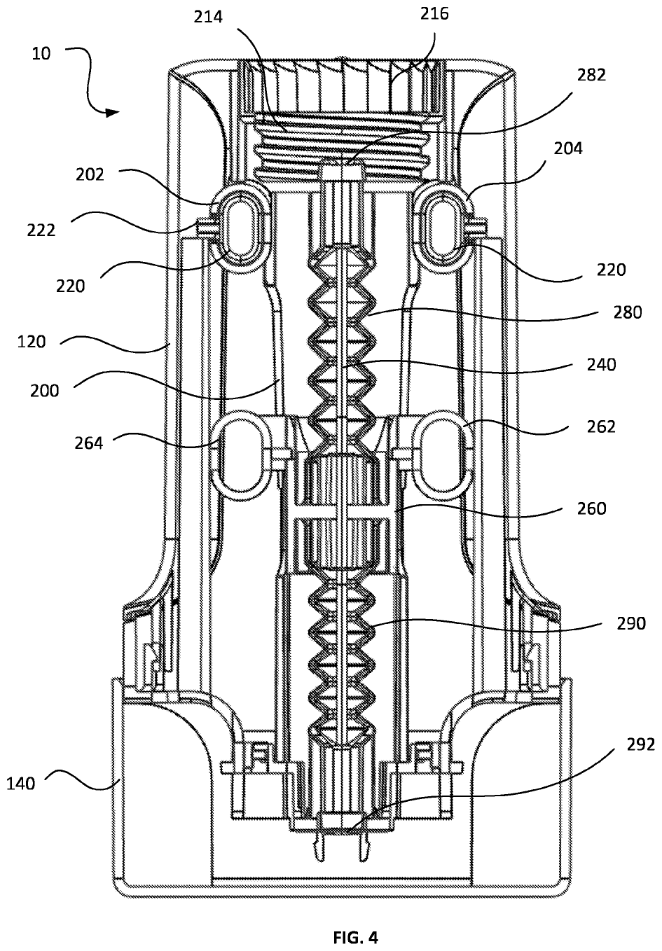

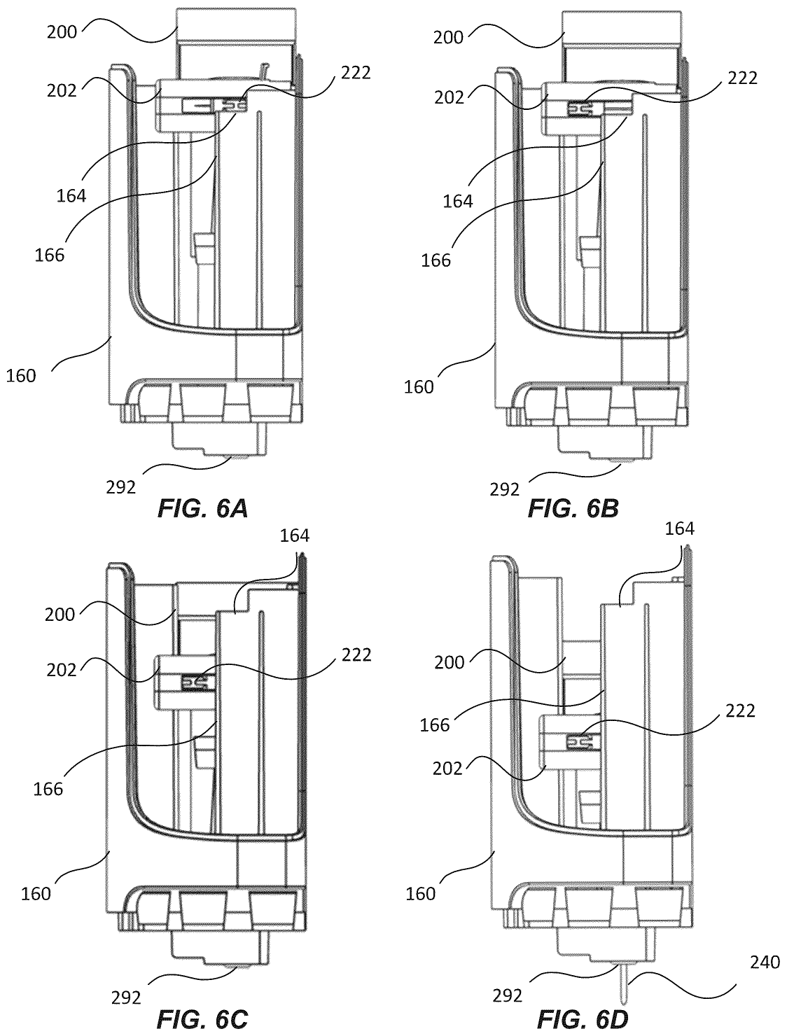

With further reference to FIGS. 5A-5C, the actuation of the actuating mechanism and the operation of the locking mechanism during use is illustrated. In particular, as shown in these figures, the clips 220 (see FIG. 4) of the hollow lugs 202, 204 are moved laterally, within the lugs 202, 204 and the protrusion 222 moving within each slot 206, 208. The clips 220 (see FIG. 4) are thus moved between a first configuration (FIG. 5A) and a second configuration (FIG. 5B). As described in more detail with respect to FIGS. 6A-6D, in the second configuration (FIG. 5B), the moveable sleeve portion 200 is enabled to longitudinally translate downwardly, toward the proximal end of the connector, to enabling piercing of the first pierceable seal 282 by the needle 240 (see FIG. 4).

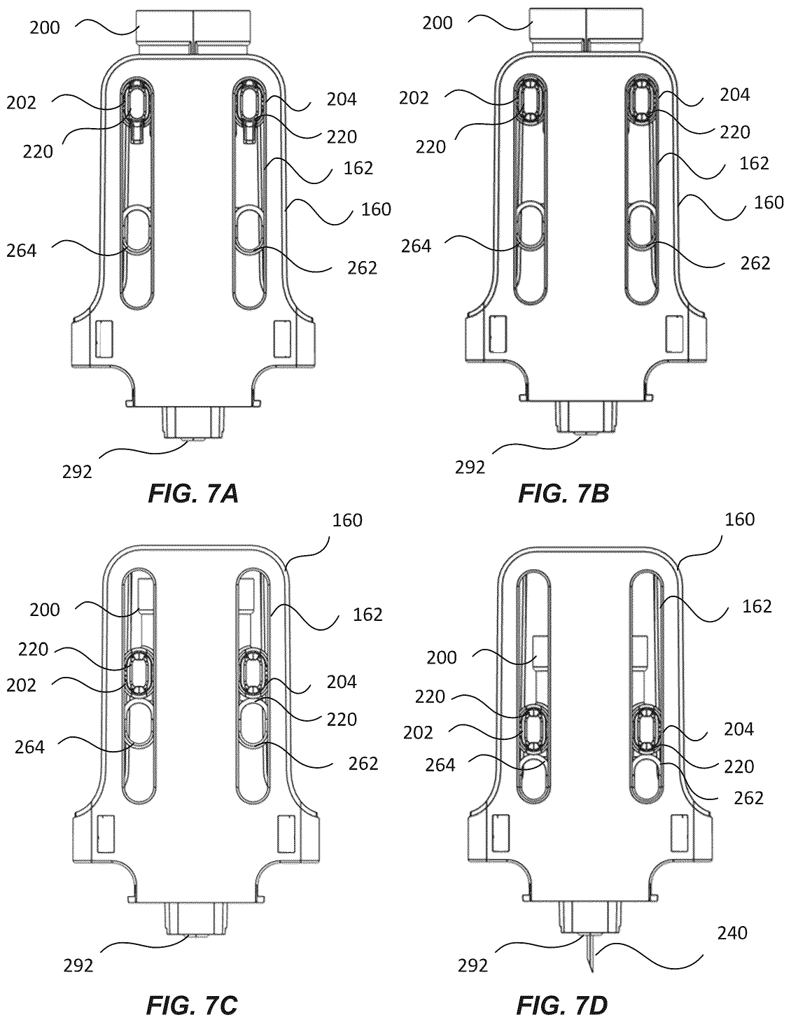

Referring now to FIGS. 6A-6D and FIGS. 7A-7D, the actuation of the connector 10 shall be described. The connector 10 is provided in a locked state, also known as the first configuration, as shown in FIGS. 6A and 7A. As shown in FIG. 6A, the protrusion 222 of the clips 220 (see FIGS. 2 to 4) are in engagement with a shoulder 164 of the stationary sleeve portion 160. In this configuration, the moveable sleeve portion 200 cannot move longitudinally with respect to the stationary sleeve portion 160, since the protrusion 222 causes a locking against the shoulder 164.

As shown in FIGS. 6B and 7B, the clips 220 are laterally moved within the lugs 202, 204, thereby moving the protrusion 222 along the slot 206, 208. In this way, the protrusion 222 is disengaged from the shoulder 164 of the stationary sleeve portion 160. The protrusion 222 is thus positioned in a longitudinal slot 166 and thus does not prevent longitudinal translation of the moveable sleeve portion 200.

As shown in FIGS. 6C and 7C, the moveable sleeve portion 200 is translated in an axial direction toward the proximal end of the connector 10, i.e., downwardly, thereby allowing the hollow needle 240, which is held static during the motion, to pierce through the first pierceable seal 282 (see FIG. 4). This provides a connection to a received container at the distal end of the connector 10, within the container-receiving portion 122 (see FIG. 1).

As shown in FIGS. 6D and 7D, the moveable sleeve portion 200 is continued in its axial trajectory, and, simultaneously, the collar 260 is axially translated toward the proximal end of the connector 10, i.e., downwardly. In this way, the hollow needle 240 continues to pierce the first pierceable seal 282, i.e., that of the moveable sleeve portion 200, and is also caused to pierce through the second pierceable seal 292, i.e., that of the stationary sleeve portion 160, thereby providing communication through the connector 10. As such, although not shown, two receptacles are connected, each at an end of the connector 10.

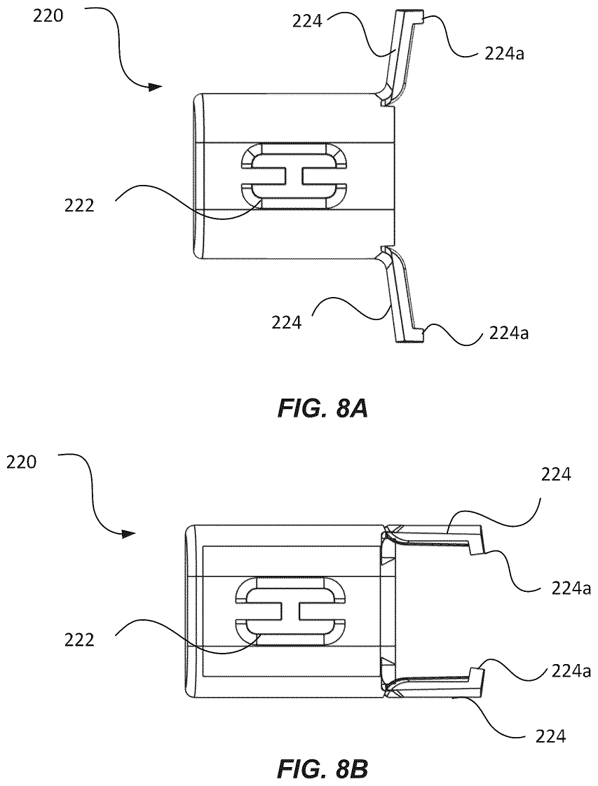

FIGS. 8A and 8B illustrate a detailed view of the clips 220, which can be provided within the hollow lugs 202, 204, 262, 264 of the moveable sleeve portion 200 and/or the collar 260. As illustrated, the clips 220 include a protrusion 222 that extends substantially perpendicular to the body of the clip 220. There are also provided, at one end of the clip 220, a pair of spring arms 224 acting as a biasing means. During use, the spring arms 224 are compressed as the clip 220 moves within the lugs 202, 204 (see FIGS. 5A and 5B), and are compressed into the configuration shown in FIG. 8B. In the configuration shown in FIG. 8B, the spring arms 224 are compressed and thus the spring force biases the clip 220 toward the first configuration (i.e., shown in FIG. 8A). Hence, once a force acting upon the clip 220 moving it to the second configuration is removed, the clip 220 is biased back into the first configuration. The spring arms 224 are also each provided with a lip 224a to provide a gripping surface to a portion of an activation system as described in further detail below. Alternatively, or in addition, the lip 224a may be arranged to engage with a recess or groove in a portion of an activation system. In this way, the clip 220 may be withdrawn by the activation system upon disengagement of the locking mechanism. Additionally, this interaction may provide tactile feedback to a user when installing/uninstalling the connector onto the activation system.

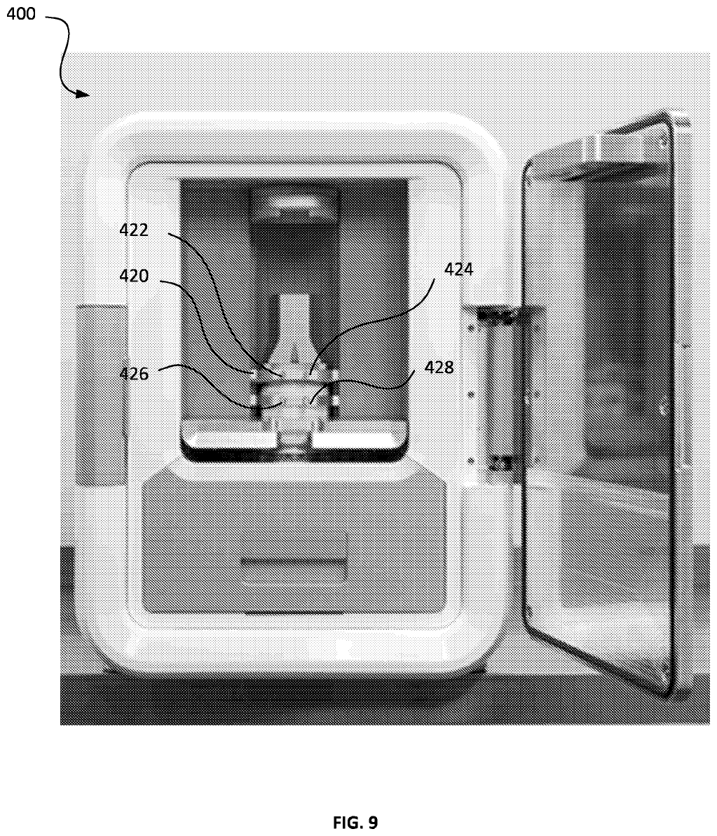

FIG. 9 illustrates an activation system 420 for activating the connector 10, particularly the locking mechanism and actuating mechanism thereof, during use. As shown in FIG. 9, an activation system 420 is provided as part of an incubator apparatus 400. The activation system 420 includes a first pair of prongs 422, 424 and a second pair of prongs 426, 428. The first part of prongs 422, 424 are configured to engage the lugs 202, 204 of the moveable sleeve portion 200, and to also engage with the clips 220 received therein. In particular, as a user installs the connector 10 onto the activation system 420, the first pair of prongs 422, 424 move the clips 220 from the first configuration into the second configuration. Each prong 422, 424 may include a recess or a groove (not shown) arranged to engage a lip 224a of the clips 220 (see FIGS. 8A and 8B) so as to engage between the prongs 422, 424 and the clip 220, and also provide tactile feedback to a user when installing/uninstalling a connector 10 onto the activation system 420. Thereafter, the activation system 420 is operable to move the moveable sleeve portion 200, i.e., cause axial translation thereof, so as to cause piercing of the first pierceable seal 282 as described above.

Additionally, the activation system 420 includes a second pair of prongs 426, 428 which are configured to engage the lugs 262, 264 of the collar 260. The second pair of prongs 426, 428 may equally engage any clips within such lugs 262, 264. Thereafter, the activation system 420 is operable to move the collar 260, i.e., cause axial translation thereof, so as to cause piercing of the second pierceable seal 292 as described above.

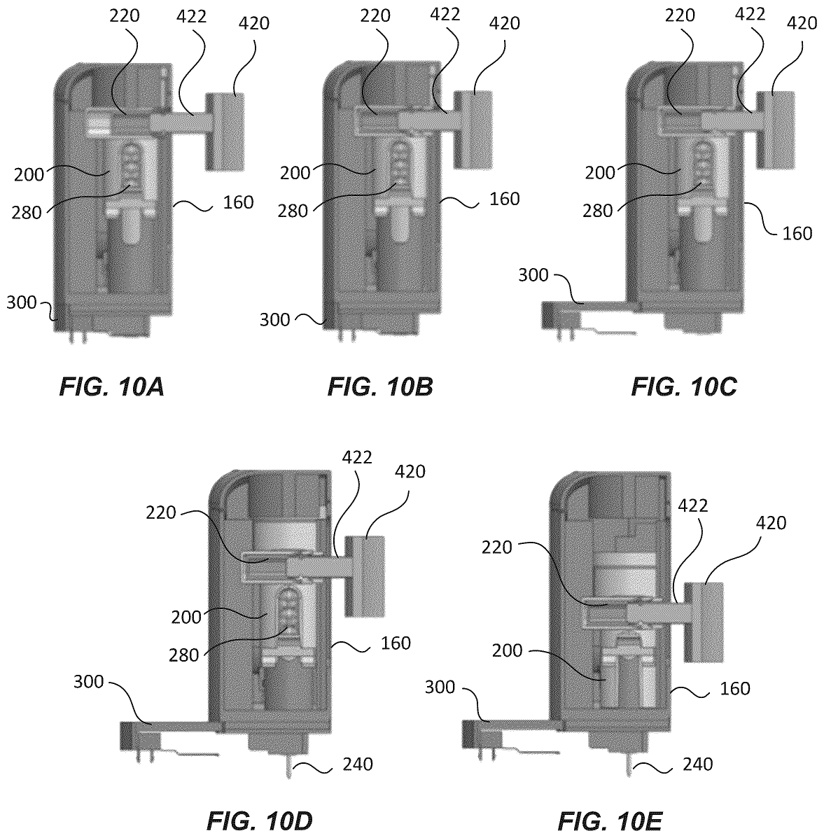

FIGS. 10A-10E illustrate the use of the activation system 420 for activating and operating the connector 10 during use. FIG. 10A illustrates the activation system 420, particularly the prong 422 engaging with a clip 220 in one of the hollow lugs of the moveable sleeve portion 200. The gaiter 280 covering the needle, and the sliding drawer 300 are also shown. Referring further to FIGS. 8A and 8B, the prong 422 engages the clip 220 such that a lip 224a of the clip 220 engages a recess or groove of the prong 422 as the spring arms 224 collapse inwardly during engagement. In FIG. 10A, the clip 220 remains in the first configuration. FIG. 10B illustrates the prong 422 inserted into the lug of the moveable sleeve portion 200 so as to move the clip 220 into the second configuration for movement (see FIGS. 6A-6D and 7A-7D, above). FIG. 10C illustrates an actuation of the sliding drawer 300 so as to remove the aseptic seal (see FIG. 2) from the second pierceable seal 292 prior to the needle being actuated. The activation system may provide a force to the sliding drawer 300 so as to move it into the position illustrated. FIG. 10D illustrates the prong 422 acting upon the moveable sleeve portion 200 so as to translate the same downwardly, i.e., toward a proximal end of the connector, and thus pierce the first pierceable seal with the needle (see FIGS. 6A-6D and 7A-7D). FIG. 10E illustrates the continued trajectory of the movement, with the prongs 426, 428 (see FIG. 9) also acting upon the collar (not shown) so as to move the hollow needle 240 through the second pierceable seal, as described in more detail above. Material can then be transferred through the connector. Once the transfer has taken place, the steps may be followed in reverse so as to allow the connector to be removed from the activation system.

As will be appreciated by persons skilled in the art, the components of the connectors, receptacles, interface place and the like may be composed of any suitable material. For example: the housing may be composed of polycarbonate, acrylonitrile butadiene styrene (ABS), high impact polystyrene sheet (HIPS); the aseptic seal composed of polyethylene (PE); the collar may be composed of polycarbonate, acrylonitrile butadiene styrene (ABS), high impact polystyrene sheet (HIPS); the hollow needle may be composed of stainless steel or another appropriate biocompatible material or metal and may have any suitable gauge; the gaiters may be composed of low density polyethylene (LDPE), a thermoplastic elastomer (TPE), a silicon or the like; the gaiter clips may be composed of high density polyethylene (HDPE); the stationary sleeve portion may be composed of polycarbonate, acrylonitrile butadiene styrene (ABS), high impact polystyrene sheet (HIPS); the pierceable seal of the stationary sleeve portion may be composed of a thermoplastic elastomer, or a silicon and/or may be co-molded with the stationary sleeve portion; the moveable sleeve portion may be composed of polycarbonate, acrylonitrile butadiene styrene (ABS), high impact polystyrene sheet (HIPS); the pierceable hermetic seal of the upper housing portion may be composed of a thermoplastic elastomer (TPE), or a silicon and/or may be co-molded with the moveable sleeve portion; and the cap may be composed of polypropylene (PP), low density polyethylene (LDPE), or a combination thereof. Any combination of materials is also envisaged as discussed above. Such materials provide multiple manufacturing and processing advantages, in addition to being suitable for cell and/or gene therapy manufacture.

Generally, it will be appreciated by persons skilled in the art that the above embodiments have been described by way of an example only and not in any limitative sense, and that various alternations and modifications are possible without departing from the scope of the invention as defined by the appended claims. Various modifications to the detailed designs as described above are possible, for example, variations may exist in shape, size, arrangement, assembly, sequence or the like.

1. A connector, for introducing or extracting material to or from at least one receptacle, comprising:
a housing, including a first end and a second end;
a hollow needle mounted, at least partially, within the housing between the first end and the second end of the housing, a first end of the hollow needle being connected or connectable to a first receptacle, and a second end of the hollow needle facing a pierceable seal;
an actuating mechanism configured to cause relative movement between the hollow needle and the pierceable seal to enable the hollow needle to pierce the pierceable seal, thereby forming a communication therethrough; and
a locking mechanism operable between a first configuration, in which the locking mechanism prevents the actuating mechanism from causing relative movement between the hollow needle and the pierceable seal, and a second configuration, in which the locking mechanism permits the actuating mechanism to cause relative movement between the hollow needle and the pierceable seal.

2. The connector according to claim 1, wherein the actuating mechanism comprises a collapsible sleeve assembly, wherein at least part of the collapsible sleeve assembly is collapsible between the first end and the second end of the housing to enable the hollow needle to pierce the pierceable seal, thereby forming the communication therethrough.

3. The connector according to claim 2, wherein the collapsible sleeve assembly comprises a moveable sleeve portion and a stationary sleeve portion, the moveable sleeve portion being axially moveable, along a central longitudinal axis of the housing, with respect to the stationary sleeve portion so as to collapse with respect thereto.

4. The connector according to claim 3, wherein the moveable sleeve portion comprises the pierceable seal.

5. The connector according to claim 3, wherein the locking mechanism comprises a clip slidably disposed within a lateral slot of the moveable sleeve portion, wherein, in the first position, the moveable clip is engaged with a portion of the stationary sleeve portion, and in the second position, the moveable clip is disengaged from the portion of the stationary sleeve portion.

6. The connector according to claim 5, wherein the clip is biased toward the first position by a biasing element.

7. The connector according to claim 6, wherein the clip comprises at least one spring arm.

8. The connector according to claim 1, wherein the hollow needle is a double-ended hollow needle.

9. The connector according to claim 4, wherein the stationary sleeve portion comprises a pierceable seal, wherein the hollow needle is a double-ended hollow needle, and wherein the double-ended hollow needle is disposed between each pierceable seal.

10. The connector according to claim 9, wherein the actuating mechanism is configured to cause relative movement between the double-ended hollow needle and each of the pierceable seals, to enable the double-ended hollow needle to pierce each of the pierceable seals, thereby forming a communication through each pierceable seal.

11. The connector according to claim 1, wherein the actuating mechanism further comprises a collar operably coupled to the hollow needle, wherein the collar is axially moveable along a central longitudinal axis.

12. The connector according to claim 11, wherein the locking mechanism is operable between a first configuration, in which the locking mechanism prevents translation of the collar along the central longitudinal axis, and a second position, in which the locking mechanism permits translation of the collar along the central longitudinal axis.

13. The connector according to claim 12, wherein the locking mechanism comprises a clip slidably disposed within a lateral slot of the collar, wherein, in the first position, the clip is engaged with a portion of a stationary sleeve portion, and in the second position, the moveable clip is disengaged from the portion of the stationary sleeve portion.

14. The connector according to claim 13, wherein the clip is biased toward the first position by a biasing element.

15. The connector according to claim 14, wherein the clip comprises at least one spring arm.

16. A system, comprising:
a connector according comprising,
a housing including a first end and a second end;
a hollow needle mounted, at least partially, within the housing between the first end and the second end of the housing, a first end of the needle being connected or connectable to a first receptacle, and a second end of the hollow needle facing a pierceable seal,
an actuating mechanism configured to cause relative movement between the hollow needle and the pierceable seal to enable the hollow needle to pierce the pierceable seal, thereby forming a communication therethrough; and
a locking mechanism operable between a first configuration, in which the locking mechanism prevents the actuating mechanism from causing relative movement between the hollow needle and the piercable seal, and a second configuration, in which the locking mechanism permits the actuating mechanism to cause relative movement between the hollow needle and the pierceable seal; and
an activation system configured to operably engage with the locking mechanism to enable operation of the locking mechanism between the first configuration and the second configuration.

17. The system of claim 16, wherein the activation system is further configured to actuate the actuating mechanism to enable the hollow needle to pierce the pierceable seal and form a communication therethrough.
